(19) United States
(12) Patent Application Publication
(10) Pub. No.: US 2019/0139391 A1
(43) Pub. Date: May 9, 2019

(54) SAFETY STATUS SENSING SYSTEM AND SAFETY STATUS SENSING METHOD THEREOF

(71) Applicant: Institute For Information Industry, Taipei (TW)

(72) Inventors: Jia-Ru LEE, Kaohsiung City (TW); Hung-Sheng CHIU, Taipei City (TW); Chih-Hao HSU, Taipei City (TW); Hsiu-Fen LEE, Nantou County (TW); Hsiao-Chen CHANG, Taipei City (TW)

(21) Appl. No.: 15/936,352

(22) Filed: Mar. 26, 2018

(30) Foreign Application Priority Data
Nov. 3, 2017 (TW) 106138149

(51) Int. Cl. G08B 21/04; A61B 5/00

(52) U.S. Cl. CPC G08B 21/0453; A61B 5/024; A61B 5/0022; A61B 5/746

(57) ABSTRACT

A safety status sensing system and a safety status sensing method thereof are provided. A first wearable sensing device periodically transmits a first safety status message to the LoRa host. After determining no message received from the first wearable sensing device within a first time period, the LoRa host issues a first alarm message and determines an off-line location of the first wearable sensing device according to the first device information of the first wearable sensing device. The LoRa host determines that the off-line location is within a communication coverage of the second wearable sensing device, and transmits the first device information to the second wearable sensing device. The second wearable sensing device transmits a point-to-point communication signal to the first wearable sensing device according to the first device information.

### PRIORITY

This application claims priority to Taiwan Patent Application No. 106138149 filed on Nov. 3, 2017, which are hereby incorporated by reference in its entirety.

### FIELD

The present invention relates to a safety status sensing system and a safety status sensing method thereof. More particularly, the present invention relates to a safety status sensing system and a safety status sensing method thereof that feature an easy deployment, a low power and a high flexibility in safety status reporting.

### BACKGROUND

In the prior art, wearable electronic devices have been widely used in various fields. One primary application of the wearable electronic devices is to sense a physiological status of a human body and report the information measured to a base station or a backhaul host for recording and analysis. In this way, it can be confirmed whether the body of the user is healthy and whether any abnormal status occurs can be determined instantly.

However, for the aforesaid wearable electronic devices and the systems thereof for detecting the safety status of the user, the network environment and the system hardware thereof usually need to be constructed in advance, and the coverage thereof in which the user can be detected is mainly based on the number of base stations in the network.

Accordingly, when a large-scale activity (e.g., a road running activity, a mountain climbing activity) is held and the aforesaid wearable electronic devices and the safety status sensing system thereof are to be used for enhancing the security of the activity, constructing the network environment and the system hardware in advance is hard to be implemented and the hardware cost required by a larger communication coverage also increases remarkably.

According to the above descriptions, an urgent need exists in the art to improve the aforesaid problem, thereby improving the flexibility and convenience in network construction of the wearable electronic devices and the safety status sensing system thereof and meanwhile reducing the construction cost.

### SUMMARY

The disclosure includes a safety status sensing method for a safety status sensing system. The safety status sensing system can comprise a LoRa host, a first wearable sensing device and a second wearable sensing device. The LoRa host communicates with the first wearable sensing device and the second wearable sensing device according to a LoRa protocol. The safety status sensing method comprises: (a) enabling the first wearable sensing device to periodically transmit a first safety status message to the LoRa host; (b) enabling the LoRa host to update first device information of the first wearable sensing device that is stored in the LoRa host according to the first safety status message; (c) enabling the LoRa host to issue a first alarm message after the LoRa host determines that no message has been received from the first wearable sensing device within a first time period; (d) enabling the LoRa host to determine an off-line location of the first wearable sensing device according to the first device information stored after the step (c); (e) enabling the LoRa host to determine that the off-line location is located within a communication coverage of the second wearable sensing device according to second device information of the second wearable sensing device, wherein the second device information is stored in the LoRa host; (f) enabling the LoRa host to transmit the first device information to the second wearable sensing device; and (g) enabling the second wearable sensing device to transmit a point-to-point communication signal to the first wearable sensing device according to the first device information.

The disclosure also includes a safety status sensing system, which comprises a LoRa host, a first wearable sensing device and a second wearable sensing device. The LoRa host can comprise a host processor, a host LoRa protocol transceiver, a host storage unit and an alarm prompting unit. The first wearable sensing device comprises a first device processor and a first device LoRa protocol transceiver. The second wearable sensing device comprises a second device processor and a second device LoRa protocol transceiver. The first wearable sensing device is configured to use the first device processor to periodically transmit a first safety status message to the LoRa host via the first device LoRa protocol transceiver. Thereafter, the LoRa host is configured to use the host LoRa protocol transceiver to receive the first safety status message from the first wearable sensing device; use the host processor to update first device information of the first wearable sensing device that is stored in the host storage unit according to the first safety status message; use the host processor to issue a first alarm message via the alarm prompting unit after the host processor determines that no message has been received by the host LoRa protocol transceiver from the first wearable sensing device within a first time period; use the host processor to determine an off-line location of the first wearable sensing device according to the first device information; use the host processor to determine that the off-line location is located within a communication coverage of the second wearable sensing device according to second device information of the second wearable sensing device, wherein the second device information is stored in the host storage unit; and use the host processor to transmit the first device information to the second wearable sensing device via the host LoRa protocol transceiver. The second wearable sensing device is configured to use the second device LoRa protocol transceiver to receive the first device information; and use the second device processor to transmit a point-to-point communication signal to the first wearable sensing device according to the first device information via the second device LoRa protocol transceiver.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1A to FIG. 1B are schematic views illustrating the operation of a safety status sensing system according to a first embodiment of the present invention;

FIG. 1C is a block diagram of a LoRa host according to the first embodiment of the present invention;

FIG. 1D is a block diagram of a first wearable sensing device according to the first embodiment of the present invention;

FIG. 1E is a block diagram of a second wearable sensing device according to the first embodiment of the present invention;

FIG. 2A to FIG. 2B are schematic views illustrating the operation of a safety status sensing system according to a second embodiment of the present invention;

FIG. 2C is a block diagram of a LoRa host according to the second embodiment of the present invention;

FIG. 2D is a block diagram of a first wearable sensing device according to the second embodiment of the present invention;

FIG. 2E is a block diagram of a second wearable sensing device according to the second embodiment of the present invention;

FIG. 2F is a block diagram of another implementation of the LoRa host according to the second embodiment of the present invention;

FIG. 2G is a block diagram of another implementation of the first wearable sensing device according to the second embodiment of the present invention;

FIG. 3 is a flowchart diagram of a safety status sensing method according to a third embodiment of the present invention; and

FIG. 4A to FIG. 4C are flowchart diagrams of a safety status sensing method according to a fourth embodiment of the present invention.

### DETAILED DESCRIPTION

In the following description, the present invention will be explained with reference to certain example embodiments thereof. However, these example embodiments are not intended to limit the present invention to any specific examples, embodiments, environment, applications or implementations described in these example embodiments. Therefore, description of these example embodiments is only for purpose of illustration rather than to limit the present invention.

In the following embodiments and the attached drawings, elements unrelated to the present invention are omitted from depiction; and dimensional relationships among individual elements in the attached drawings are illustrated only for ease of understanding, but not to limit the actual scale.

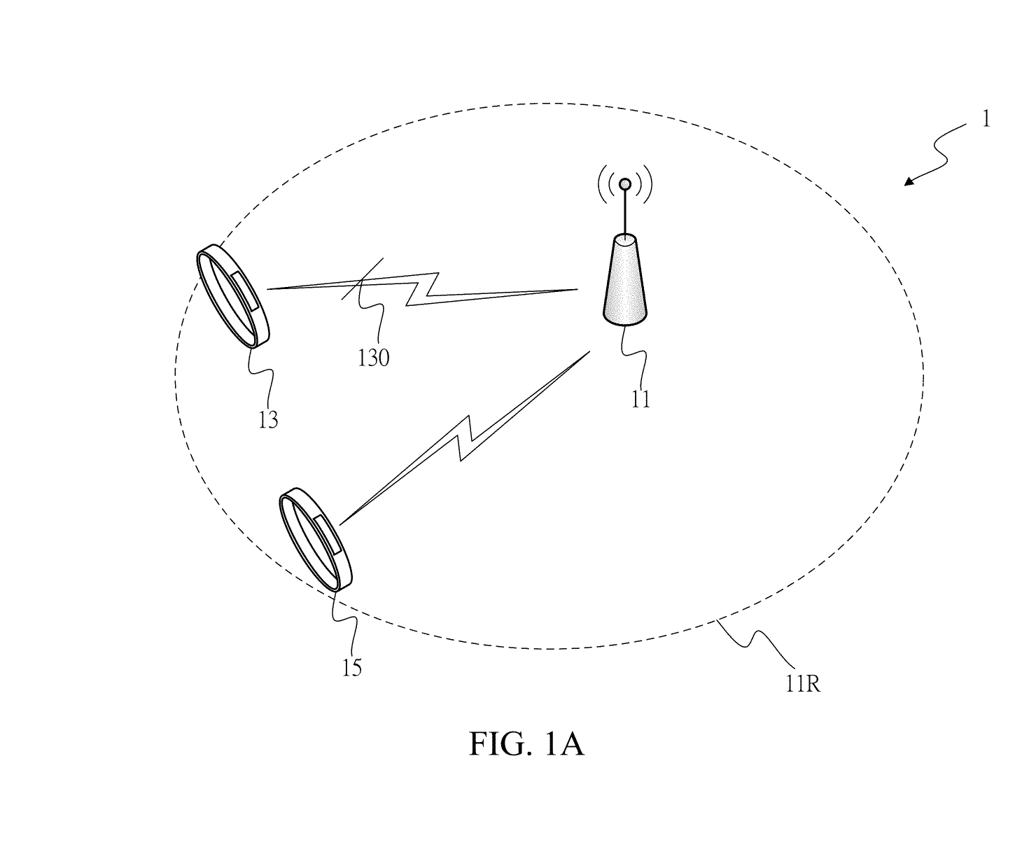

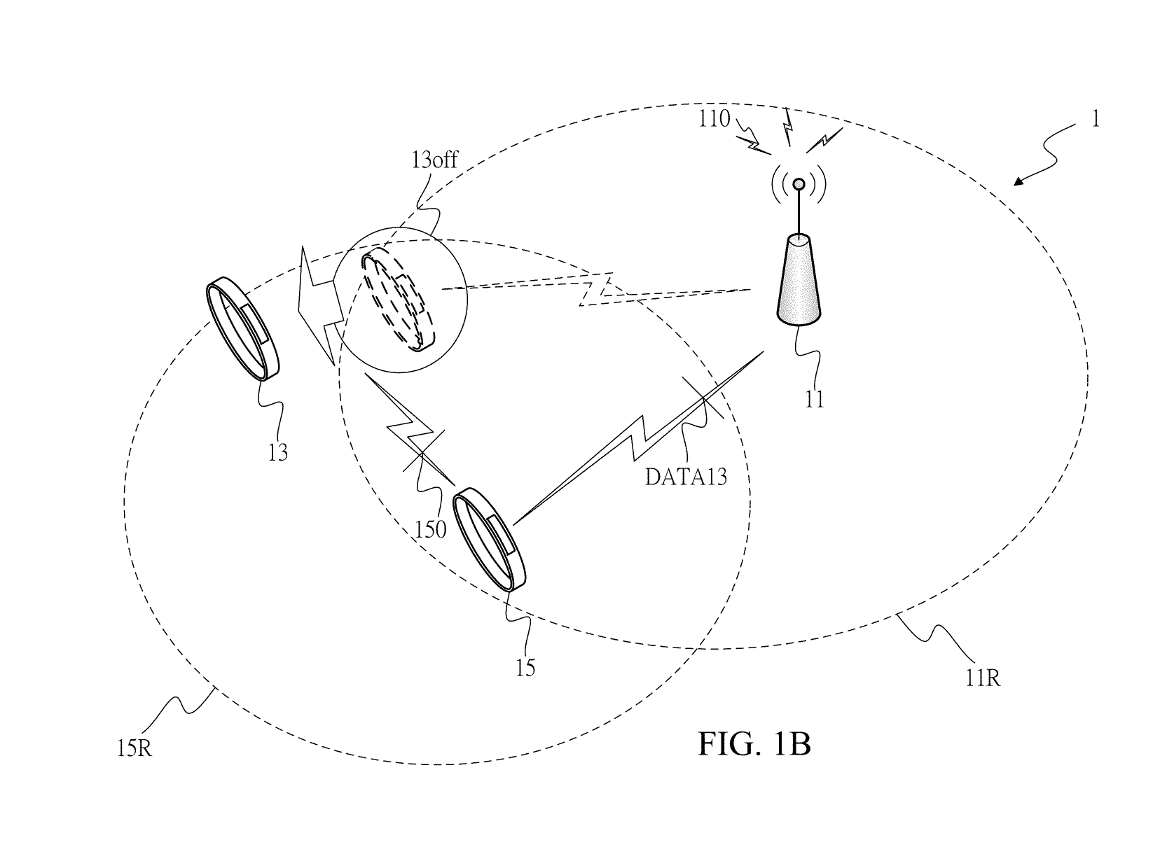

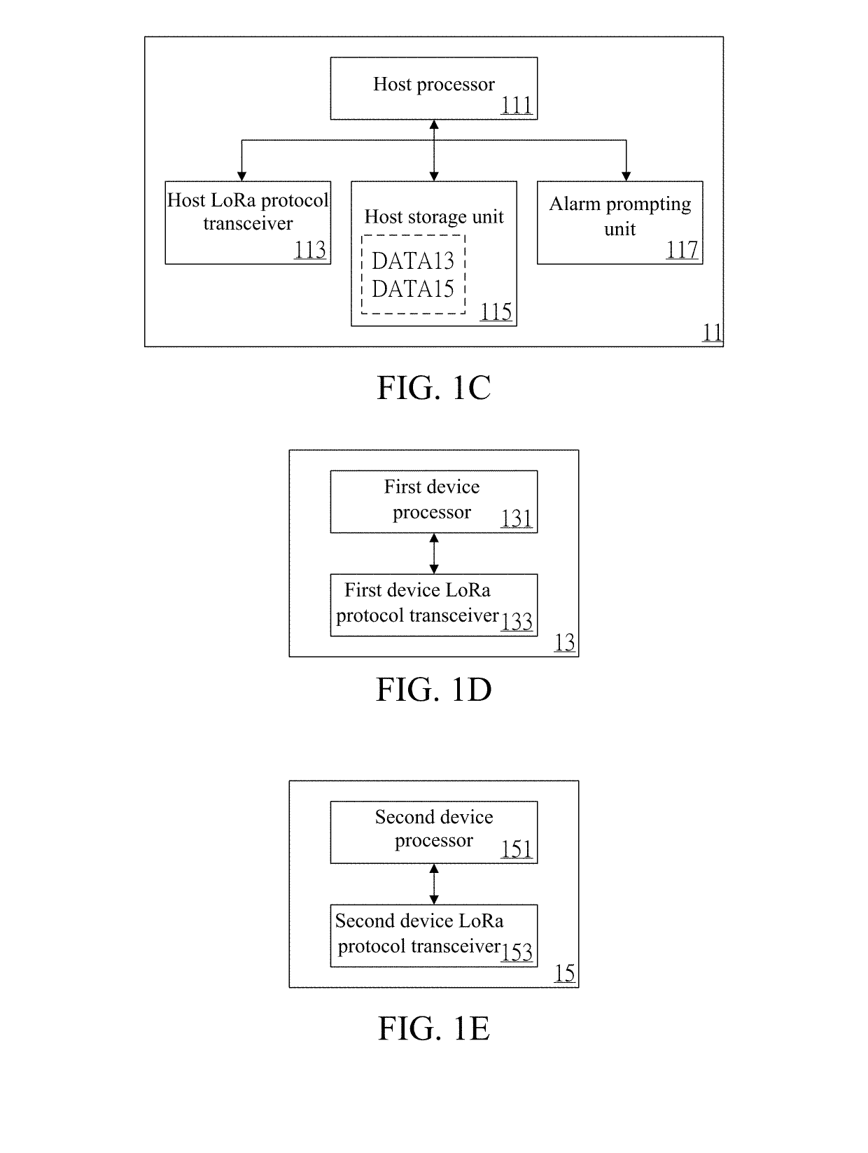

Please refer to FIG. 1A to FIG. 1E. FIG. 1A to FIG. 1B are schematic views illustrating the operation of a safety status sensing system 1 according to a first embodiment of the present invention. The safety status sensing system 1 comprises a LoRa host 11, a first wearable sensing device 13 and a second wearable sensing device 15. FIG. 1C is a block diagram of the LoRa host 11 according to the first embodiment of the present invention, and the LoRa host 11 comprises a host processor 111, a host LoRa protocol transceiver 113, a host storage unit 115 and an alarm prompting unit 117.

FIG. 1D is a block diagram of the first wearable sensing device 13 according to the first embodiment of the present invention, and the first wearable sensing device comprises a first device processor 131 and a first device LoRa protocol transceiver 133. FIG. 1E is a block diagram of the second wearable sensing device 15 according to the first embodiment of the present invention, and the second wearable sensing device comprises a second device processor 151 and a second device LoRa protocol transceiver 153. Elements of each of the devices are electrically connected with each other, and the devices communicate with each other via the LoRa communication protocol. Interactions among the devices will be further described hereinafter.

First referring to FIG. 1A, in the first embodiment, the first wearable sensing device 13 and the second wearable sensing device 15 are respectively worn by different users and periodically report information relevant to user safety to the LoRa host 11. Specifically, taking the first wearable sensing device 13 as an example, the first wearable sensing device 13 is mainly configured to periodically transmit a first safety status message 130 relevant to the user safety status to the LoRa host 11 via the first device LoRa protocol transceiver 133.

On the other hand, when the first wearable sensing device 13 is still within a communication coverage 11R of the LoRa host 11, the host LoRa protocol transceiver 113 of the LoRa host 11 can receive the first safety status message 130 from the first wearable sensing device 13, and the host processor 111 updates first device information DATA13 relevant to the first wearable sensing device 13 that is stored in the host storage unit 115 according to the first safety status message 130 so as to master safety information relevant to the user of the first wearable sensing device 13 instantly. Similarly, the host storage unit 115 stores second device information DATA15 of the second wearable sensing device 15 (in other embodiments, it may further comprise device information of other wearable sensing devices).

Then referring to FIG. 1B, when the user of the first wearable sensing device 13 moves so that the first wearable sensing device 13 is not within the communication coverage 11R of the LoRa host 11, the LoRa host 11 cannot receive any safety status message from the first wearable sensing device 13, so the LoRa host 11 cannot monitor the safety status of the user of the first wearable sensing device 13.

In other words, if the host processor 111 of the LoRa host 11 determines that no message has been received from the first wearable sensing device 13 by the host LoRa protocol transceiver 113 within a first time period (not shown), then it means that concerns have been raised over the safety detection of the user. Thus, the host processor 111 of the LoRa host 11 first issues a first alarm message 110 via the alarm prompting unit 117.

Thereafter, the host processor 111 of the LoRa host 11 first determines an off-line location 13off of the first wearable sensing device 13 according to the first device information DATA13 that is updated latest and stored in the host storage unit 115. Next, the LoRa host 11 searches for other wearable sensing devices that are around the off-line location 13off of the first wearable sensing device 13 and within the communication coverage 11R of the LoRa host 11 according to the off-line location 13off and the second device information DATA15 (in other implementations, other device information is further comprised) stored in the host storage unit 115, in order to attempt to connect with the first wearable sensing device 13 via the other wearable sensing devices.

In the first embodiment, the host processor 111 of the LoRa host 11 determines that the off-line location 13off is located within a communication coverage 15R of the second wearable sensing device 15 according to the second device information DATA15 of the second wearable sensing device 15 that is stored in the host storage unit 115. Accordingly, the host processor 111 transmits the first device information DATA13 to the second wearable sensing device 15 via the host LoRa protocol transceiver 113 so that the second wearable sensing device 15 attempts to connect with the first wearable sensing device 13.

On the other hand, after the second device LoRa protocol transceiver 153 of the second wearable sensing device 15 receives the first device information DATA13, the second device processor 151 transmits a point-to-point communication signal 150 to the first wearable sensing device 13 via the second device LoRa protocol transceiver 153 according to the first device information DATA13, thereby attempting to contact with the first wearable sensing device 13.

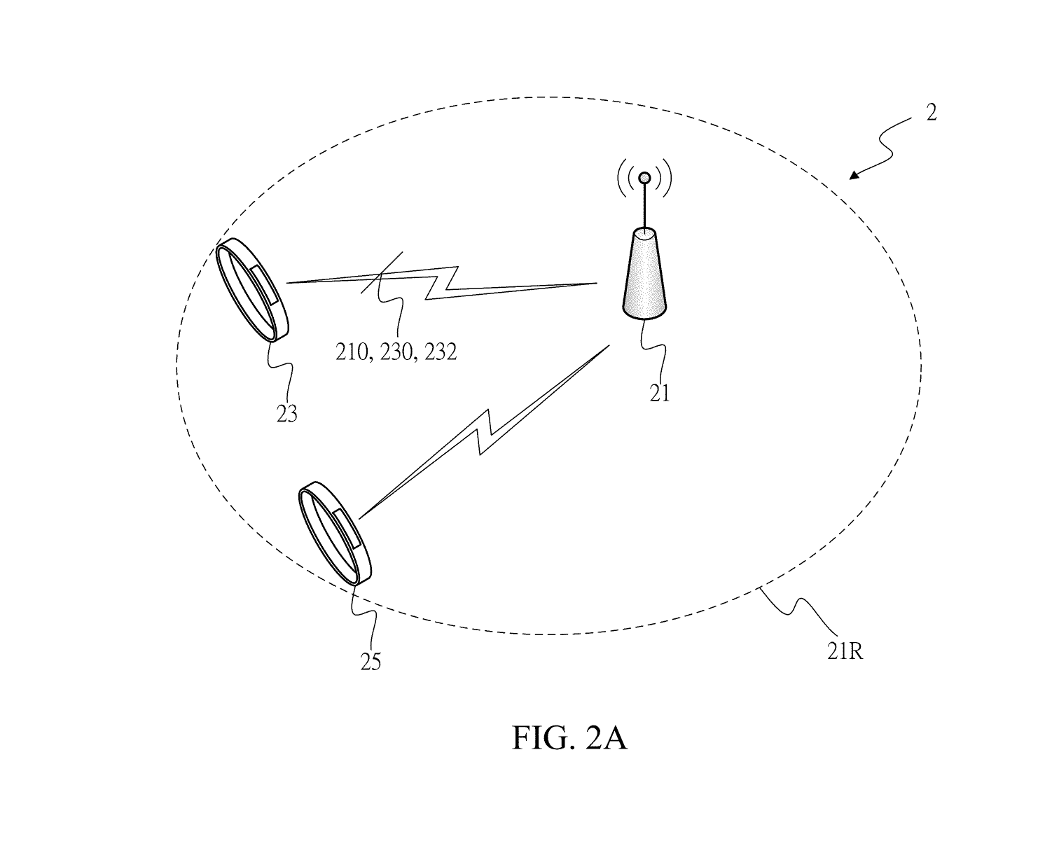

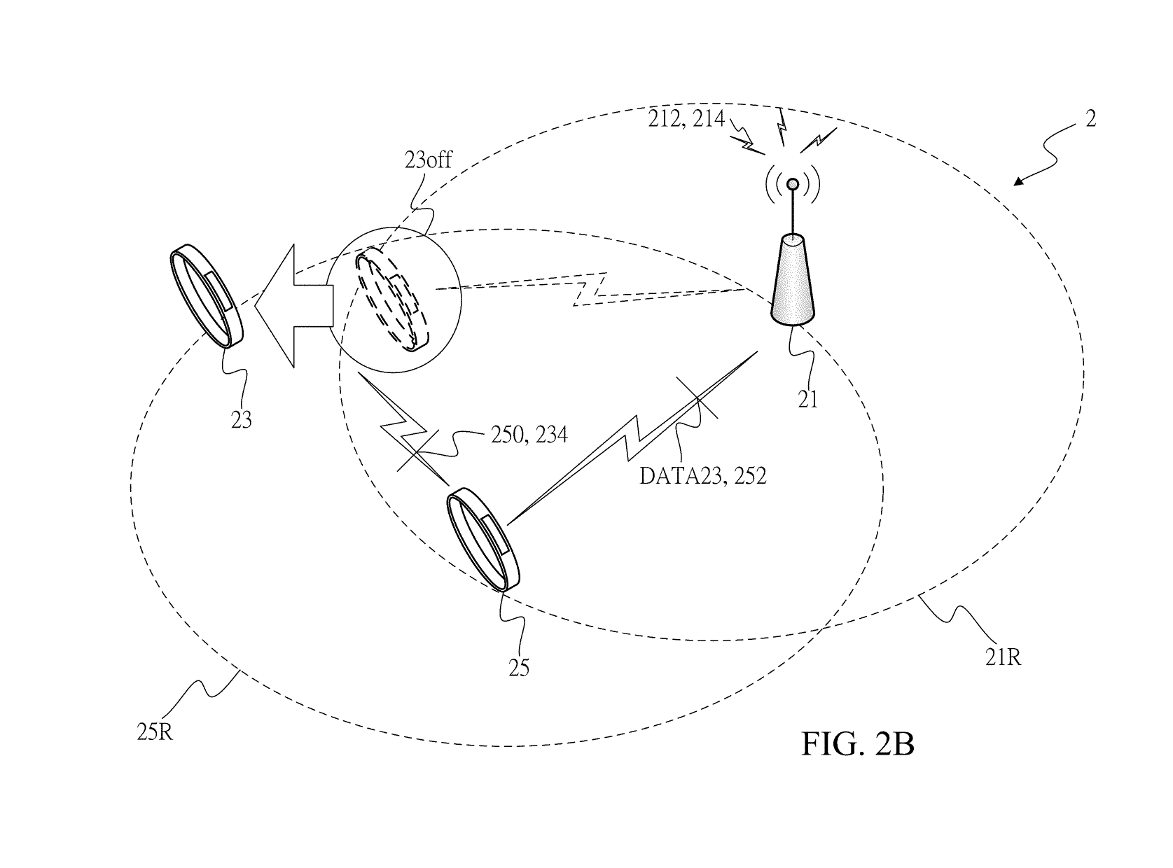

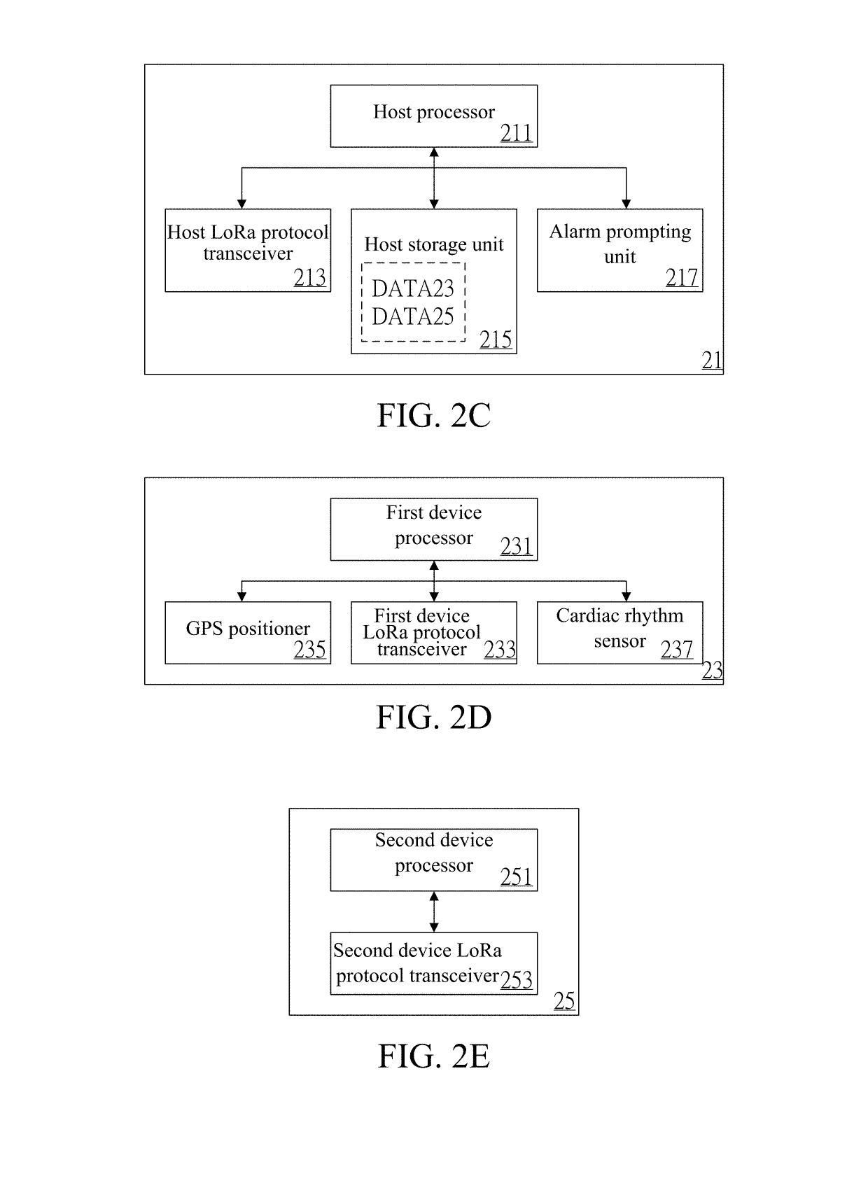

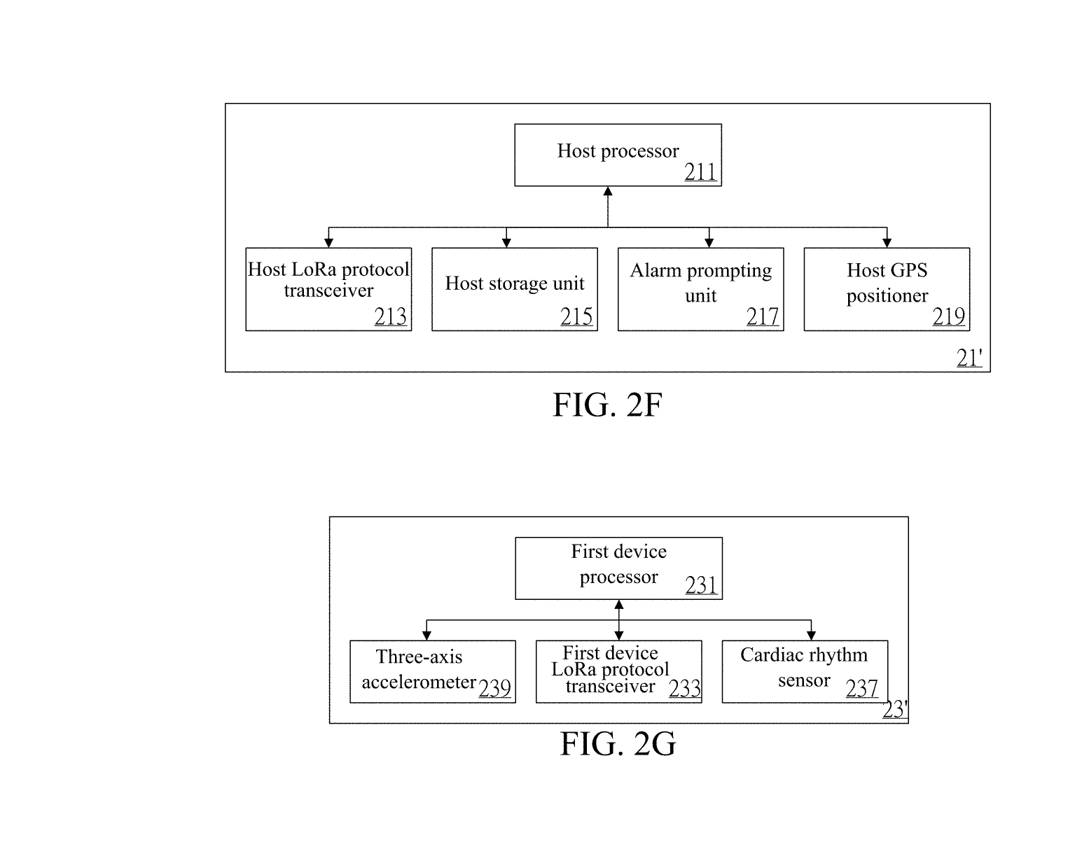

Please refer to FIG. 2A to FIG. 2E. FIG. 2A to FIG. 2B are schematic views illustrating the operation of a safety status sensing system 2 according to a second embodiment of the present invention. The safety status sensing system 2 comprises a LoRa host 21, a first wearable sensing device 23 and a second wearable sensing device 25. FIG. 2C is a block diagram of a LoRa host 21 according to the second embodiment of the present invention, and the LoRa host 21 comprises a host processor 211, a host LoRa protocol transceiver 213, a host storage unit 215 and an alarm prompting unit 217.

FIG. 2D is a block diagram of a first wearable sensing device according to the second embodiment of the present invention, and the first wearable sensing device comprises a first device processor 231, a first device LoRa protocol transceiver 233, a first GPS positioner 235 and a first cardiac rhythm sensor 237. FIG. 2E is a block diagram of the second wearable sensing device 25 according to the second embodiment of the present invention, and the second wearable sensing device comprises a second device processor 251 and a second device LoRa protocol transceiver 253.

Similarly, elements of each of the devices in the aforesaid second embodiment are electrically connected with each other, and the devices communicate with each other via the LoRa communication protocol. The second embodiment mainly further illustrates operation details and subsequent detection statuses of the safety status sensing system of the present invention. It shall be particularly appreciated that, because the second wearable sensing device 25 of the second embodiment is mainly used as a relay device when the first wearable sensing device 23 is disconnected from the LoRa host 21, only the main elements (the second device processor 251 and the second device LoRa transceiver 253) are illustrated and the positioning or heart beat sensing elements thereof are not particularly described. However, this is not intended to limit the implementation of the second wearable sensing device 25.

First referring to FIG. 2A, in the second embodiment, during the deployment of the safety status sensing system 2, the host LoRa protocol transceiver 213 of the LoRa host 21 broadcasts an initial setting message 210 to notify wearable sensing devices in the network environment to report information, thereby initializing network environment parameters. On the other hand, after receiving the initial setting message 210, the first device LoRa protocol transceiver 233 of the first wearable sensing device 21 can report to the LoRa host 21.

In detail, the first device processor 231 of the first wearable sensing device 23 reports an initial status message 230 to the LoRa host 21 via the first device LoRa protocol transceiver 233 according to the initial setting message 210 so as to inform the LoRa host 21 of the status of the first wearable sensing device 23. After the host LoRa protocol transceiver 213 of the LoRa host 21 receives the initial status message 230, the host processor 211 accordingly decides first device information DATA23 of the first wearable sensing device 23 and records the first device information DATA23 into the host storage unit 215. Similarly, the host processor 211 records second device information DATA25 of the second wearable sensing device 25 into the host storage unit 215 (in other implementations, the host storage unit 215 may further comprise device information of other wearable sensing devices).

Next, in the second embodiment, the first wearable sensing device 23 and the second wearable sensing device 25 are respectively worn by different users and periodically report information relevant to user safety to the LoRa host 21. Specifically, taking the first wearable sensing device 23 as an example, the first wearable sensing device 23 is mainly configured to periodically transmit a first safety status message 232 relevant to the user safety status to the LoRa host 21 via the first device LoRa protocol transceiver 233.

On the other hand, when the first wearable sensing device 23 is still within a communication coverage 21R of the LoRa host 21, the host LoRa protocol transceiver 213 of the LoRa host 21 can receive the first safety status message 230 from the first wearable sensing device 23, and the host processor 211 updates the first device information DATA23 relevant to the first wearable sensing device 23 that is stored in the host storage unit 215 so as to master safety information relevant to the user of the first wearable sensing device 23 instantly.

It shall be particularly appreciated that, in the second embodiment, the first wearable sensing device 23 has a first device identification (not shown), and the first safety status message 232 reported to the LoRa host 21 periodically by the first wearable sensing device 23 comprises the first device identification (ID), a first heart beat rate detected by the first cardiac rhythm sensor 237, a first time and a first location detected by the first GPS positioner 235. Accordingly, the first device information DATA23 is mainly configured to periodically record and update the first device ID, the first heart beat rate, the first time and the first location associated with the first wearable sensing device 23.

Then referring to FIG. 2B, when the user of the first wearable sensing device 23 moves so that the first wearable sensing device 23 is not within the communication coverage 21R of the LoRa host 21, the LoRa host 21 cannot receive any safety status message from the first wearable sensing device 23, so the LoRa host 21 cannot monitor the safety status of the user of the first wearable sensing device 23.

In other words, if the host processor 211 of the LoRa host 21 determines that no message has been received from the first wearable sensing device 23 by the host LoRa protocol transceiver 213 within a first time period (not shown), then it means that concerns have been raised over the safety detection of the user. Thus, the host processor 211 of the LoRa host 21 first issues a first alarm message 212 via the alarm prompting unit 217.

Thereafter, the host processor 211 of the LoRa host 21 first determines an off-line location 23off of the first wearable sensing device 23 according to the first device information DATA23 that is updated latest and stored in the host storage unit 215. Next, the LoRa host 21 searches for other wearable sensing devices that are around the off-line location of the first wearable sensing device 23 and within the communication coverage 21R of the LoRa host 21 according to the off-line location 23off and the second device information DATA25 stored in the host storage unit 215, in order to attempt to connect with the first wearable sensing device 23 via the other wearable sensing devices.

In the second embodiment, the host processor 211 of the LoRa host 21 determines the location and the communication coverage of the second wearable sensing device 25 according to the second device information DATA25 of the second wearable sensing device 25 that is stored in the host storage unit 215, and thus it may further determine that the off-line location 23off is located within a communication coverage 25R of the second wearable sensing device 25. Accordingly, the host processor 211 transmits the first device information DATA23 to the second wearable sensing device 25 via the host LoRa protocol transceiver 213 so that the second wearable sensing device 25 attempts to connect with the first wearable sensing device 23.

On the other hand, after the second device LoRa protocol transceiver 253 of the second wearable sensing device 25 receives the first device information DATA23, the second device processor 251 transmit a point-to-point communication signal 250 to the first wearable sensing device 23 via the second device LoRa protocol transceiver 253 according to the first device information DATA23, thereby attempting to contact with the first wearable sensing device 23.

More particularly, in the second embodiment, the second device processor 251 of the second wearable sensing device 25 determines via the second device LoRa protocol transceiver 253 whether any response message corresponding to the point-to-point communication signal 250 is received from the first wearable sensing device 23 within a second time period (not shown) after the point-to-point communication signal 250 is transmitted.

If the second device processor 251 of the second wearable sensing device 25 determines that no response message is received from the first wearable sensing device 23 within the second time period, then the concern that the user of the first wearable sensing device 23 is in an unsafe status is remarkably increased. Thus, the second device LoRa protocol transceiver 253 of the second wearable sensing device 25 transmits a no-response message 252 to the LoRa host 21. After the host LoRa protocol transceiver 213 receives the no-response message 252, the host processor 211 of the LoRa host 21 issues a second alarm message 214 via the alarm prompting unit 217.

On the other hand, if the second device processor 251 of the second wearable sensing device 25 determines that a response message 234 corresponding to the point-to-point communication signal 250 is received from the first wearable sensing device 23 within the second time period, then the second device processor 251 transmits via the second device LoRa protocol transceiver 253 the response message 234 to the LoRa host 21. The response message 234 comprises information relevant to the safety of the first wearable sensing device 23, so the host processor 211 of the LoRa host 21 can accordingly update the first device information DATA23 stored in the LoRa host storage unit 215 for subsequent tracing of the user safety information after the host LoRa protocol transceiver 213 receives the response message 234.

It shall be additionally appreciated that, in other implementations, the location of the wearable sensing device may be decided via other hardware. Specifically, please refer to FIG. 2F and FIG. 2G. FIG. 2F is a block diagram of another implementation of a LoRa host 21′ according to the second embodiment of the present invention, and the LoRa host 21′ comprises a host processor 211, a host LoRa protocol transceiver 213, a host storage unit 215, an alarm prompting unit 217 and a host GPS positioner 219. FIG. 2G is a block diagram of another implementation of a first wearable sensing device 23′ according to the second embodiment of the present invention, and the first wearable sensing device 23′ comprises the first device processor 231, the first device LoRa protocol transceiver 233, a three-axis accelerometer 239 and a first cardiac rhythm sensor 237.

Further speaking, in other implementations, during the deployment of the safety status sensing system 2, the GPS positioner 219 of the LoRa host 21 may first generate a host GPS location (not shown) and transmit the host GPS location to the first wearable sensing device 23 via the host LoRa protocol transceiver 213. Accordingly, when the first wearable sensing device 23 knows the initial location of the LoRa host 21 and starts to move away from the LoRa host 21, the three-axis accelerometer 239 of the first wearable sensing device 23 starts to generate displacement information (not shown). In this case, the first device processor 231 can calculate the first location based on the initial location (i.e., the host GPS location) and the displacement information, and update the first location at any moment according to the displacement information generated by the three-axis accelerometer 239. In this way, the location of the wearable sensing device can be estimated and updated even without the GPS positioner.

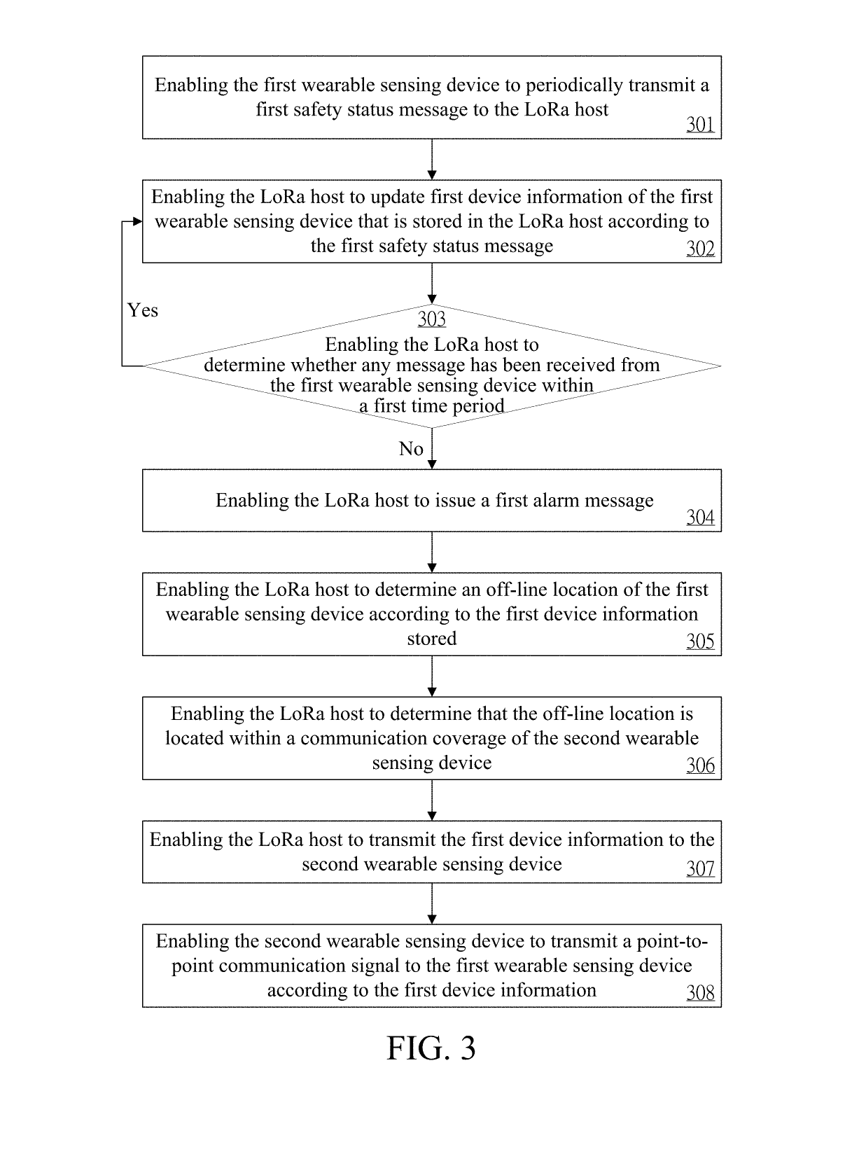

A third embodiment of the present invention is a safety status sensing method, and a flowchart diagram thereof is as shown in FIG. 3. The method of the third embodiment is for use in a safety status sensing system (e.g., the safety status sensing system of the aforesaid embodiments). The safety status sensing system comprises a LoRa host, a first wearable sensing device and a second wearable sensing device. The LoRa host communicates with the first wearable sensing device and the second wearable sensing device according to a LoRa protocol. Detailed steps of the third embodiment are as follows.

First, step 301 is executed to enable the first wearable sensing device to periodically transmit a first safety status message to the LoRa host. Step 302 is executed to enable the LoRa host to update first device information of the first wearable sensing device that is stored in the LoRa host according to the first safety status message. Step 303 is executed to enable the LoRa host to determine whether any message has been received from the first wearable sensing device within a first time period. If the determination result is yes, then the step 302 is executed again.

On the other hand, if the determination result is no, then step 304 is executed to enable the LoRa host to issue a first alarm message. Next, step 305 is executed to enable the LoRa host to determine an off-line location of the first wearable sensing device according to the first device information stored. Step 306 is executed to enable the LoRa host to determine that the off-line location is located within a communication coverage of the second wearable sensing device according to second device information of the second wearable sensing device. The second device information is stored in the LoRa host.

Thereafter, step 307 is executed to enable the LoRa host to transmit the first device information to the second wearable sensing device. Step 308 is executed to enable the second wearable sensing device to transmit a point-to-point communication signal to the first wearable sensing device according to the first device information so as to attempt to contact with the first wearable sensing device.

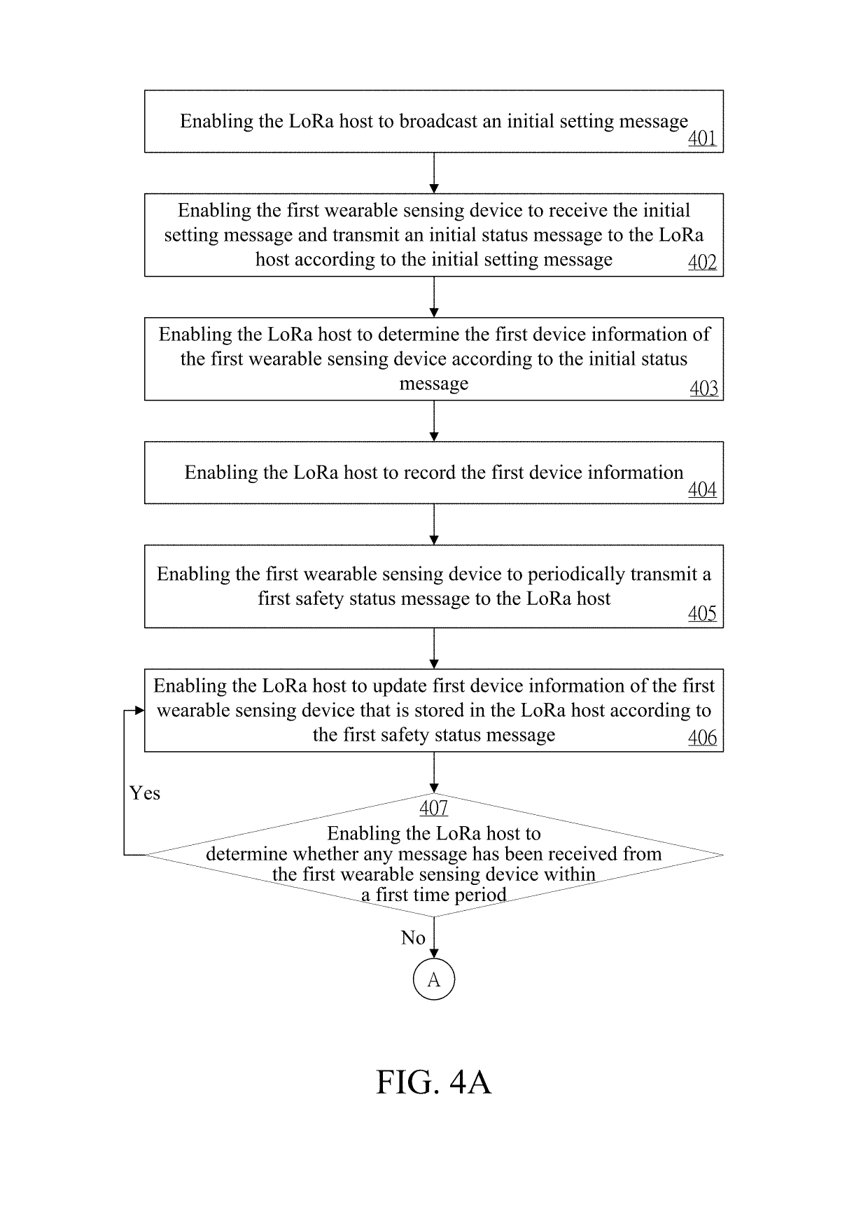

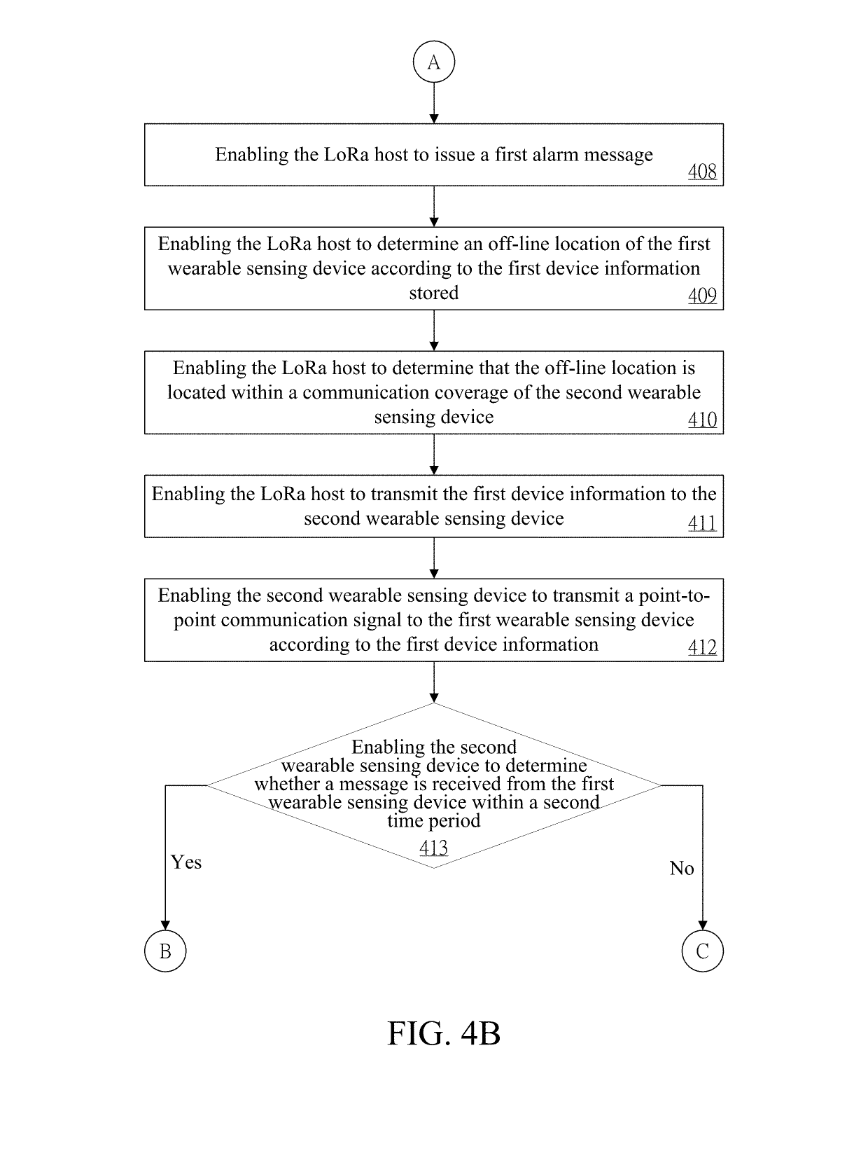

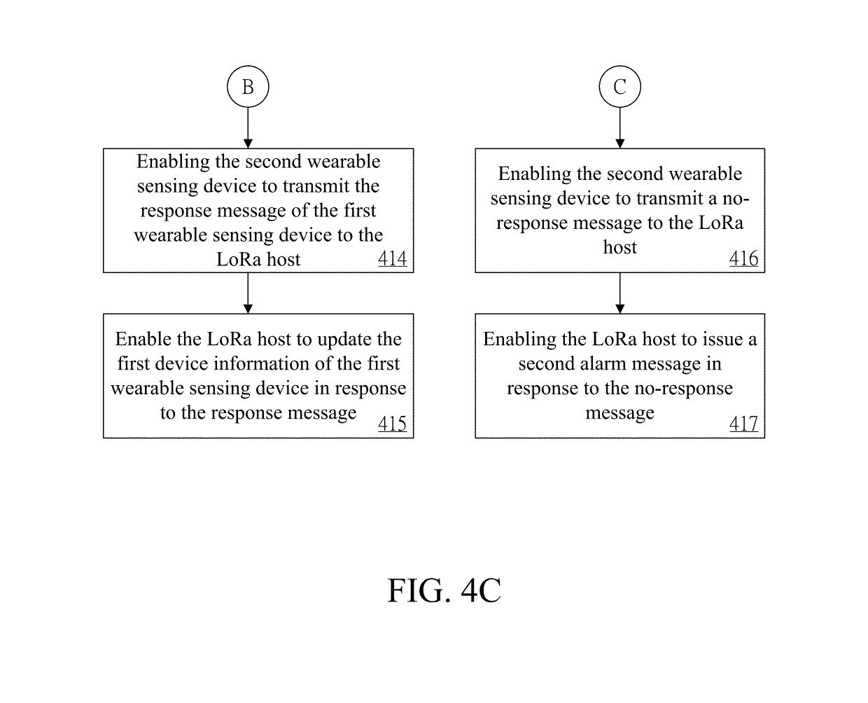

A fourth embodiment of the present invention is a safety status sensing method, and flowchart diagrams thereof are as shown in FIG. 4A to FIG. 4C. The method of the fourth embodiment is for use in a safety status sensing system (e.g., the safety status sensing system of the aforesaid embodiments). The safety status sensing system comprises a LoRa host, a first wearable sensing device and a second wearable sensing device. The LoRa host communicates with the first wearable sensing device and the second wearable sensing device according to a LoRa protocol. Detailed steps of the fourth embodiment are as follows.

First, step 401 is executed to enable the LoRa host to broadcast an initial setting message. Step 402 is executed to enable the first wearable sensing device to receive the initial setting message and transmit an initial status message to the LoRa host according to the initial setting message. Step 403 is executed to enable the LoRa host to determine the first device information of the first wearable sensing device according to the initial status message. Step 404 is executed to enable the LoRa host to record the first device information.

Next, step 405 is executed to enable the first wearable sensing device to periodically transmit a first safety status message to the LoRa host. Step 406 is executed to enable the LoRa host to update first device information of the first wearable sensing device that is stored in the LoRa host according to the first safety status message. Step 407 is executed to enable the LoRa host to determine whether any message has been received from the first wearable sensing device within a first time period. If the determination result is yes, then the step 406 is executed again.

On the other hand, if the determination result is no, then step 408 is executed to enable the LoRa host to issue a first alarm message. Next, step 409 is executed to enable the LoRa host to determine an off-line location of the first wearable sensing device according to the first device information stored. Step 410 is executed to enable the LoRa host to determine that the off-line location is located within a communication coverage of the second wearable sensing device according to second device information of the second wearable sensing device. The second device information is stored in the LoRa host.

Thereafter, step 411 is executed to enable the LoRa host to transmit the first device information to the second wearable sensing device. Step 412 is executed to enable the second wearable sensing device to transmit a point-to-point communication signal to the first wearable sensing device according to the first device information so as to attempt to contact with the first wearable sensing device. Step 413 is executed to enable the second wearable sensing device to determine whether a response message corresponding to the point-to-point communication signal is received from the first wearable sensing device within a second time period.

If the determination result is yes, then step 414 is executed to enable the second wearable sensing device to transmit the response message of the first wearable sensing device to the LoRa host. Step 415 is executed to enable the LoRa host to update the first device information of the first wearable sensing device in response to the response message. If the determination result is no, then step 416 is executed to enable the second wearable sensing device to transmit a no-response message to the LoRa host. Step 417 is executed to enable the LoRa host to issue a second alarm message in response to the no-response message.

According to the above descriptions, the safety status sensing system and the safety status sensing method of the present invention deploy the host and the wearable devices in the network environment mainly through the LoRa communication protocol of a low power, and utilize the relay characteristic of nodes in the LoRa protocol specification to extend the communication coverage for safety reporting of the nodes. In this way, the flexibility and convenience in network construction of the wearable electronic devices can be improved remarkably, and the construction cost can also be reduced effectively.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

1. A safety status sensing method for a safety status sensing system, the safety status sensing system comprising a LoRa host, a first wearable sensing device and a second wearable sensing device, and the LoRa host communicating with the first wearable sensing device and the second wearable sensing device according to a LoRa protocol, the safety status sensing method comprising:
(a) the first wearable sensing device periodically transmitting a first safety status message to the LoRa host;
(b) the LoRa host updating a first device information of the first wearable sensing device that is stored in the LoRa host according to the first safety status message;
(c) the LoRa host issuing a first alarm message after the LoRa host determines that no message has been received from the first wearable sensing device within a first time period;
(d) the LoRa host determining an off-line location of the first wearable sensing device according to the first device information stored after the step (c);
(e) the LoRa host determining that the off-line location is located within a communication coverage of the second wearable sensing device according to second device information of the second wearable sensing device, wherein the second device information is stored in the LoRa host;
(f) the LoRa host transmitting the first device information to the second wearable sensing device; and
(g) the second wearable sensing device transmitting a point-to-point communication signal to the first wearable sensing device according to the first device information.

2. The safety status sensing method of claim 1, further comprising the following steps before the step (a):
(a1) the LoRa host broadcasting an initial setting message;
(a2) the first wearable sensing device receiving the initial setting message and transmitting an initial status message to the LoRa host according to the initial setting message;
(a3) the LoRa host determining the first device information of the first wearable sensing device according to the initial status message; and
(a4) the LoRa host to recording the first device information.

3. The safety status sensing method of claim 1, further comprising:
(h) the second wearable sensing device determining, after the step (g), that no response message corresponding to the point-to-point communication signal is received from the first wearable sensing device within a second time period;
(i) the second wearable sensing device transmitting a no-response message to the LoRa host according to the result of the step (h); and
(j) the LoRa host issuing a second alarm message in response to the no-response message.

4. The safety status sensing method of claim 1, further comprising:
(h) the second wearable sensing device determining, after the step (g), that a response message corresponding to the point-to-point communication signal is received from the first wearable sensing device within a second time period;
(i) the second wearable sensing device transmitting the response message to the LoRa host according to the result of the step (h); and
(j) the LoRa host updating the first device information of the first wearable sensing device in response to the response message.

5. The safety status sensing method of claim 1, wherein the first safety status message and the first device information respectively comprise a first device identification (ID), a first heart beat rate, a first time and a first location associated with the first wearable sensing device.

6. A safety status sensing system, comprising:
a LoRa host, comprising a host processor, a host LoRa protocol transceiver, a host storage unit and an alarm prompting unit;
a first wearable sensing device, comprising a first device processor and a first device LoRa protocol transceiver; and
a second wearable sensing device, comprising a second device processor and a second device LoRa protocol transceiver;
wherein the first wearable sensing device is configured to use the first device processor to periodically transmit a first safety status message to the LoRa host via the first device LoRa protocol transceiver;
wherein the LoRa host is configured to use the host LoRa protocol transceiver to receive the first safety status message from the first wearable sensing device; use the host processor to update first device information of the first wearable sensing device that is stored in the host storage unit according to the first safety status message; use the host processor to issue a first alarm message via the alarm prompting unit after the host processor determines that no message has been received by the host LoRa protocol transceiver from the first wearable sensing device within a first time period; use the host processor to determine an off-line location of the first wearable sensing device according to the first device information; use the host processor to determine that the off-line location is located within a communication coverage of the second wearable sensing device according to second device information of the second wearable sensing device, wherein the second device information is stored in the host storage unit; and use the host processor to transmit the first device information to the second wearable sensing device via the host LoRa protocol transceiver; and
wherein the second wearable sensing device is further configured to use the second device LoRa protocol transceiver to receive the first device information; and use the second device processor to transmit a point-to-point communication signal to the first wearable sensing device according to the first device information via the second device LoRa protocol transceiver.

7. The safety status sensing system of claim 6, wherein the LoRa host is further configured to use the host LoRa protocol transceiver to broadcast an initial setting message;
wherein the first wearable sensing device is further configured to use the first device processor to receive the initial setting message and transmit an initial status message to the LoRa host according to the initial setting message via the first device LoRa protocol transceiver; and
wherein the LoRa host is further configured to use the host LoRa protocol transceiver to receive the initial status message; use the host processor to determine the first device information of the first wearable sensing device according to the initial status message; and use the LoRa host storage unit to record the first device information.

8. The safety status sensing system of claim 6, wherein the second wearable sensing device is further configured to use the second device processor to determine that no response message corresponding to the point-to-point communication signal is received by the second device LoRa protocol transceiver from the first wearable sensing device within a second time period; and use the second device LoRa protocol transceiver to transmit a no-response message to the LoRa host; and
wherein the LoRa host is further configured to use the host LoRa protocol transceiver to receive the no-response message; and use the host processor to issue a second alarm message via the alarm prompting unit in response to the no-response message.

9. The safety status sensing system of claim 6, wherein the second wearable sensing device is further configured to use the second device processor to determine that a response message corresponding to the point-to-point communication signal is received by the second device LoRa protocol transceiver from the first wearable sensing device within a second time period; and use the second device LoRa protocol transceiver to transmit the response message to the LoRa host; and
wherein the LoRa host is further configured to use the host LoRa protocol transceiver to receive the response message; and use the host processor to update the first device information of the first wearable sensing device that is stored in the LoRa host storage unit in response to the response message.

10. The safety status sensing system of claim 6, wherein the first wearable sensing device has a first device ID and further comprises:
a GPS positioner, being configured to determine a first location; and
a cardiac rhythm sensor, being configured to detect a first heart beat rate of a user;
wherein the first safety status message and the first device information respectively comprise the first device ID, the first heart beat rate, a first time and the first location.

11. The safety status sensing system of claim 6, wherein the LoRa host further comprises a GPS positioner configured to generate a host GPS location and transmit the host GPS location to the first wearable sensing device via the host LoRa protocol transceiver, and the first wearable sensing device has a first device ID and further comprises:
a three-axis accelerometer, being configured to generate displacement information; and
a cardiac rhythm sensor, being configured to detect a first heart beat rate of a user;
wherein the first device processor generates a first location according to the host GPS location and the displacement information, and the first safety status message and the first device information respectively comprise the first device ID, the first heart beat rate, a first time and the first location.

12. A first wearable sensing device, comprising:
a first device processor; and
a first device LoRa protocol transceiver;
wherein the first device processor periodically transmits a safety status message to a LoRa host via the first device LoRa protocol transceiver, the first device LoRa protocol transceiver is further configured to receive a point-to-point communication signal from a second wearable sensing device, and the first device processor is further configured to transmit a response message to the second wearable sensing device via the first device LoRa protocol transceiver according to the point-to-point communication signal.

13. The first wearable sensing device of claim 12, wherein the first device processor further receives an initial setting message of the LoRa host and transmits an initial status message to the LoRa host according to the initial setting message via the first device LoRa protocol transceiver.

14. The first wearable sensing device of claim 12, wherein the first wearable sensing device has a first device ID and further comprises:
a GPS positioner, being configured to determine a first location; and
a cardiac rhythm sensor, being configured to detect a first heart beat rate of a user;
wherein the first safety status message and the first device information respectively comprise the first device ID, the first heart beat rate, a first time and the first location.

15. The first wearable sensing device of claim 12, wherein the first wearable sensing device has a first device ID, and the first device LoRa protocol transceiver is further configured to receive a host GPS location from the LoRa host, the first wearable sensing device further comprising:
a three-axis accelerometer, being configured to generate displacement information; and
a cardiac rhythm sensor, being configured to detect a first heart beat rate of a user;
wherein the first device processor generates a first location according to the host GPS location and the displacement information, and the first safety status message and the first device information respectively comprise the first device ID, the first heart beat rate, a first time and the first location.
